(19) United States
(12) Patent Application Publication
(10) Pub. No.: US 2012/0037160 A1
(43) Pub. Date: Feb. 16, 2012

(54) RESPIRATOR AND METHOD OF IDENTIFYING CLEANLINESS/TURBIDITY OF FILTER THEREOF

(76) Inventor: Ching-Lung SUNG, Taichung City (TW)

(21) Appl. No.: 13/162,773

(22) Filed: Jun. 17, 2011

(30) Foreign Application Priority Data
Aug. 11, 2010 (TW) 99126776
Aug. 11, 2010 (TW) 99126779

(51) Int. Cl. A61M 16/00; A61M 16/20

(52) U.S. Cl. 128/205.12; 128/204.18; 128/205.24; 128/205.23

(57) ABSTRACT

A respirator includes an external shell assembly, an internal shell assembly mounted inside the external shell assembly and communicating with outside of the external shell assembly through an intake passage, and a sound-absorbing member mounted inside the internal shell assembly and defining a channel therein. An air blower can be mounted inside the internal shell assembly for sucking the oxygen or air inputted externally into the internal shell assembly along the channel and then outputting it further to where it is required. The acoustic absorption of the sound-absorbing member and the covering of the internal and external shell assemblies can effectively lower the noise of the respirator, and the internal shell assembly can further focus the gas to enhance the efficiency of suction of the gas of the air blower and to reduce the heat generated by the air blower.

### BACKGROUND OF THE INVENTION

1. Field of the Invention

The present invention relates generally to a medical apparatus, and more particularly, to a respirator.

2. Description of the Related Art

A conventional respirator is provided for the patient who suffers a particular disease, like SARS and H1N1, which disables his or her lungs from reaching sufficient gas exchange. The respirator is very common in the emergency ward or extensive care unit of the hospital. A household respirator is also available for the patient in need of long-term respiratory care at home.

Most of the popular respirators are positive pressure ones, each of which is composed of a housing, an air blower mounted inside the housing, an intake pipeline connected with the housing, and a gas supply pipeline communicating with the air blower. The air blower can suck the oxygen or air entering the housing through the intake pipeline and then convey oxygen or air to the nasal or full-face mask that the patient wears through the gas supply pipeline and finally to the patient's lungs.

However, the conventional respirator is defective because the air blower generates heat, which is not subject to dissipation, to heighten the temperature of the respirator, and makes much noise to not only interfere with the patient's rest but harass other people therearound. Besides, the conventional respirator can only provide oxygen or air without change to the gas source in communication with the intake pipeline; namely, the respirator cannot switch the gas type any time subject to the patent's need. In other words, the conventional respirator is still defective to need further improvement.

### SUMMARY OF THE INVENTION

The primary objective of the present invention is to provide a respirator, the working temperature of which is not subject to rise and the working noise of which is not much.

The foregoing objective of the present invention is attained by the respirator composed of a housing and a sound-absorbing member. The housing includes an external shell assembly defining an external space outside itself, an internal shell assembly mounted inside the external shell and defining an internal space therein, and a first intake passage for communication between the external and internal spaces. The sound-absorbing member is mounted inside the internal space and defines a channel in communication with the internal space. The internal shell assembly can receive the oxygen or air inputted externally and the oxygen or air can flow along the channel of the sound-absorbing member and be pressurized and conveyed by the air blower out of the housing further to where it is required. The sound-absorbing member can absorb the noise made while the air blower sucks the gas inside the internal shell, and by the covering of the internal and external shell assemblies, the noise conveyed outside the housing can be greatly reduced, so the overall noise of the respirator is less than that of the prior art. In addition, the internal shell assembly can further focus the gas inside the housing to enhance the efficiency of suction of the gas of the air blower and to reduce the heat generated by the air blower, so the working temperature of the air blower is not subject to increase.

The secondary objective of the present invention is to provide a respirator, which can switch gas type as per the patient's need.

The foregoing objective of the present invention is attained by the respirator, the housing of which further comprises a second intake passage for communication between the internal and external spaces. The housing further comprises a rotary valve which is pivotable between a first position, at which the first intake passage is blocked from communicating with the internal space, and a second position, at which the second intake passage is blocked from communicating with the internal space. The first and second intake passages of the housing can communicate with an oxygen source and an air source separately. When the rotary valve is located at the first position, the second intake passage can have the air be inputted into the internal space for the air blower to suck and outputted to where it is required. Similarly, when the rotary valve is located at the second position, the respirator can output the oxygen inputted into the internal space through the first intake passage.

### BRIEF DESCRIPTION OF THE DRAWINGS

FIG. 1 is a partially exploded view of a preferred embodiment of the present invention, illustrating that the lower and upper shell pieces are separated from each other.

FIG. 2 is a perspective view of the preferred embodiment of the present invention, from which the upper shell piece, the internal shell piece, and a plurality of the sound-absorbing members are removed for convenient illustration.

FIG. 3 is an exploded view of parts of the preferred embodiment of the present invention.

FIG. 4 is a sectional view of the preferred embodiment of the present invention, illustrating that the rotary valve is located at the first position.

FIG. 5 is a top view of a part of the preferred embodiment of the present invention.

FIG. 6 is a top view of parts of the preferred embodiment of the present invention.

FIG. 7 is a bottom view of a part of the preferred embodiment of the present invention.

FIG. 8 is a top view of parts of the preferred embodiment of the present invention.

FIG. 9 is similar to FIG. 4, illustrating that the rotary valve is located at the second position.

FIG. 10 is a schematic view of the preferred embodiment of the present invention, illustrating the filter cleanliness/turbidity identification system.

FIG. 11 is a flow chart of the preferred embodiment of the present invention, illustrating the method of identifying the cleanliness/turbidity of the filter.

### DETAILED DESCRIPTION OF PREFERRED EMBODIMENTS

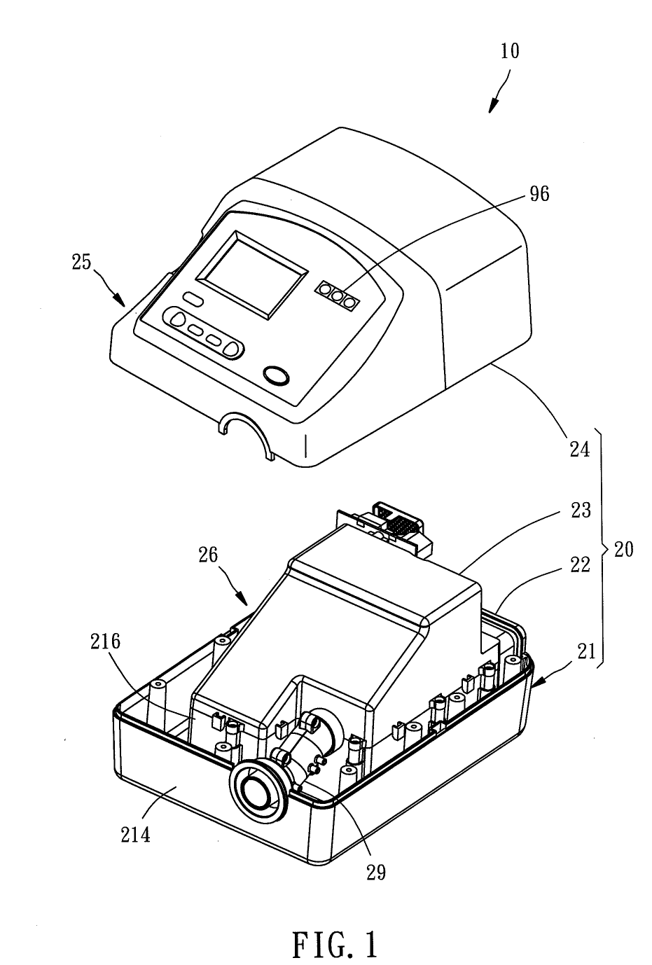

Referring to FIGS. 1-3, a respirator 10 constructed according a preferred embodiment of the present invention is composed of a housing 20, a first sound-absorbing member 30, a second sound-absorbing member 40, and an air blower 50. The detailed descriptions and operations of these elements as well as their interrelations are recited in the respective paragraphs as follows.

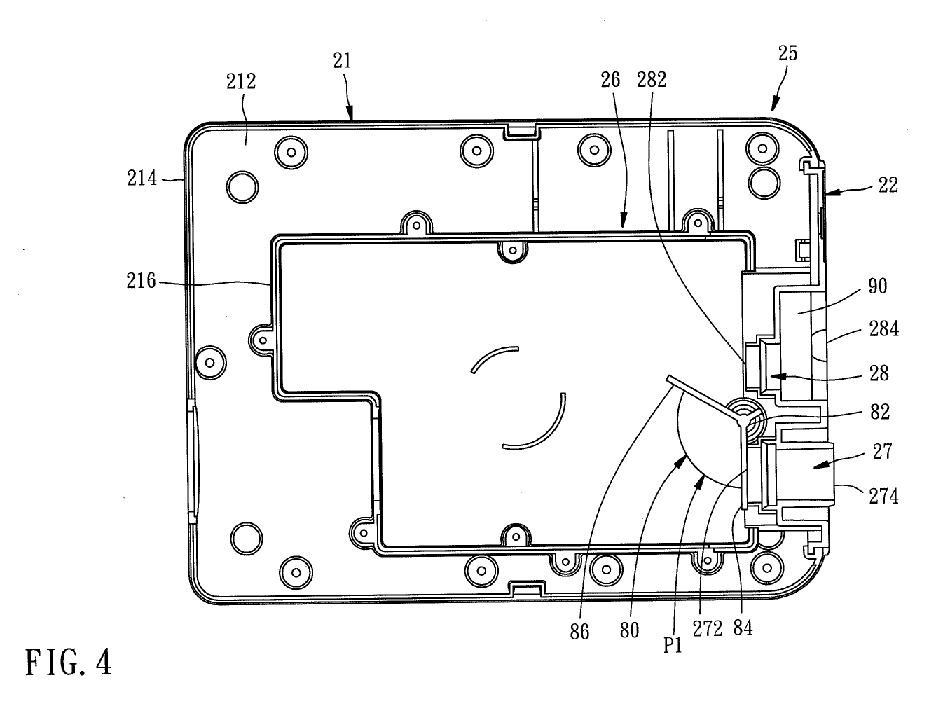

The housing 20 includes a lower shell piece 21, a rear shell piece 22, an internal shell piece 23, and an upper shell piece 24. The lower shell piece 21 has a bottom 212, as shown in FIG. 4, an external wall 214 integrally protruding upward from the bottom 212, and an internal wall 216 integrally protruding upward from the bottom 212. The rear shell piece 22 is mounted to a rear end portion of the lower shell piece 21 and connected with the external and internal walls 214 and 216. The internal shell piece 23 is mounted above the lower shell piece 21 and connected with the internal wall 216 and the rear shell piece 22. The upper shell piece 24 is mounted above the lower shell piece 21 and the internal shell piece 23 and connected with the external wall 214 and the rear shell piece 22. The upper shell piece 24, the bottom 212 of the lower shell piece 21, the external wall 214, and the rear shell piece 22 jointly constitute an external shell assembly 25 and define an external space outside the external shell assembly 25. The internal shell piece 23, the bottom 212 of the lower shell piece 21, the internal wall 216, and the rear shell piece 22 jointly constitute an internal shell assembly 26 and define an internal space inside the internal shell assembly 26. Besides, the housing 20 further includes a first intake passage 27 and a second intake passage 28, which are located at the rear shell piece 22. The first intake passage 27 is provided with a first inner opening 272 and a first outer opening 274; the former faces the internal space of the internal shell assembly 26 and the latter faces the external space of the external shell assembly 25. The second intake passage 28 is provided with a second inner opening 282 and a second outer opening 284; the former faces the internal space of the internal shell assembly 26 and the latter faces the external space of the external shell assembly 25. In other words, the first and second intake passages 27 and 28 can allow communication between the internal and external spaces.

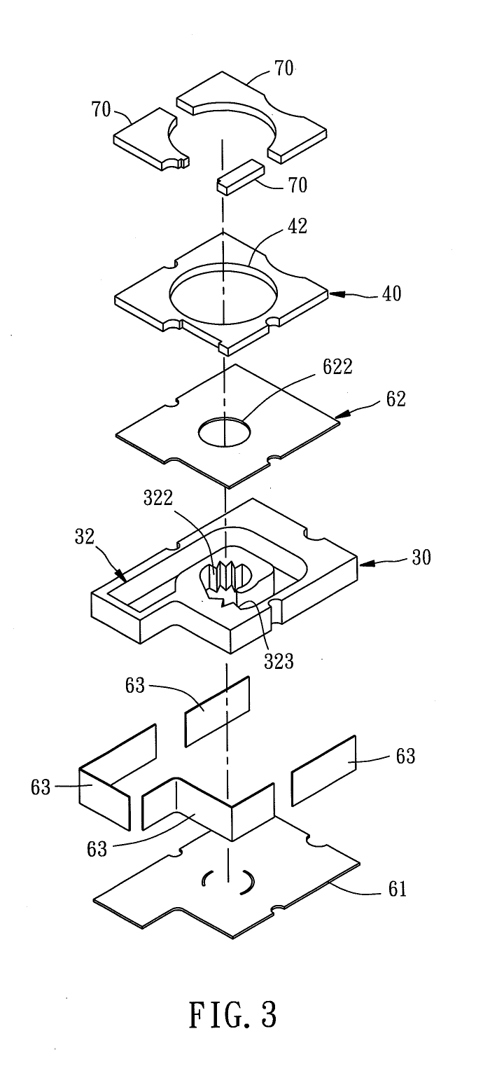

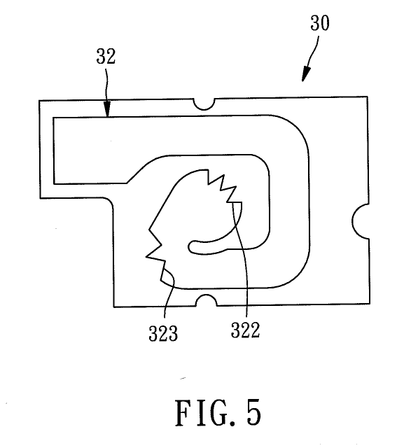

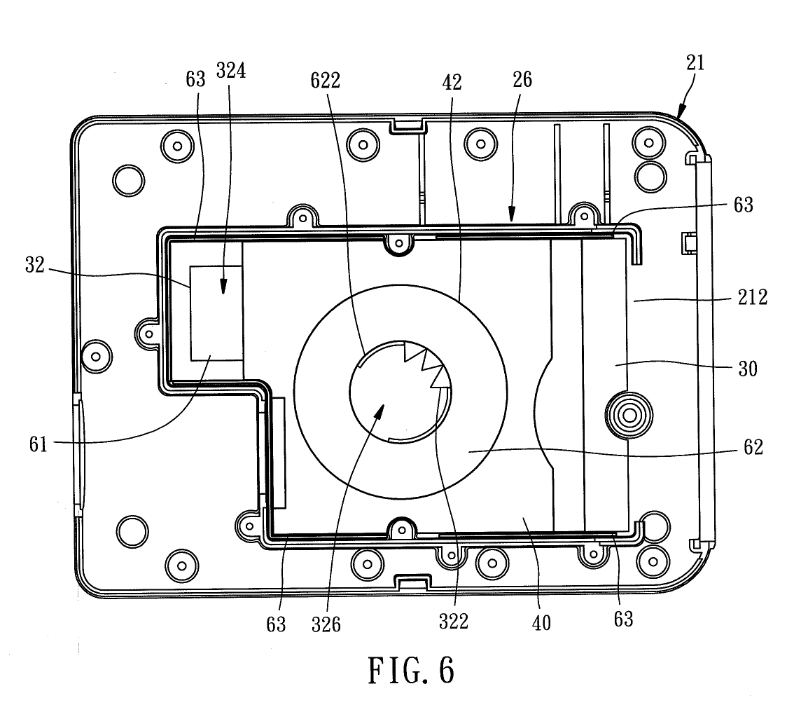

Referring to FIGS. 3-5, each of the first and second sound-absorbing members 30 and 40 is made of foam which can absorb noise. The first sound-absorbing member 30 corresponds to the internal shell assembly 26 in profile and includes a curled channel 32 running therethrough. The channel 32 is provided with a first toothed portion 322 formed at one end of a center of the first sound-absorbing member 30, and a second toothed portion 323 adjacent to the first toothed portion 322. The second sound-absorbing member 40 is smaller than the first sound-absorbing member 30 in profile and has a through hole 42 running through a center thereof. Referring to FIGS. 3 and 6, the first and second sound-absorbing members 30 and 40 are mounted inside the internal space of the shell assembly 26. In other words, the first sound-absorbing member 30 is mounted above the bottom 212 of the lower shell piece 21 and a shock-resistant bottom plate 61 is mounted between the first sound-absorbing member 30 and the bottom 212. The second sound-absorbing member 40 is located above the first sound-absorbing member 30 and a shock-resistant intermediate plate 62 is mounted between the first and second sound-absorbing members 30 and 40. The shock-resistant intermediate plate 62 is provided with an orifice 622. The shock-resistant intermediate plate 62 covers a part of the channel 32, so the channel 32 is provided with an entrance 324 and an exit 326, both of which face upward. The first toothed portion 322 is located at the exit 326. The second toothed portion 323 is adjacent to the exit 326. Furthermore, a plurality of shock-resistant standing plates 63 are mounted between the first sound-absorbing member 30 and an inner side of the internal wall 216 of the lower shell piece 21.

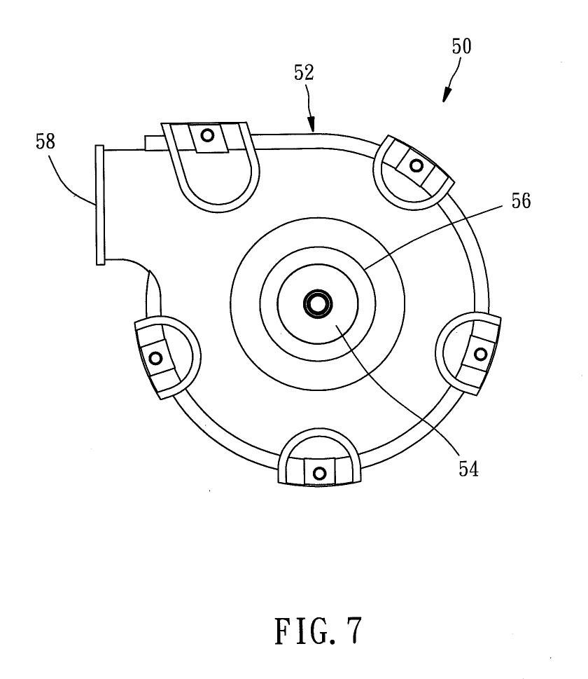

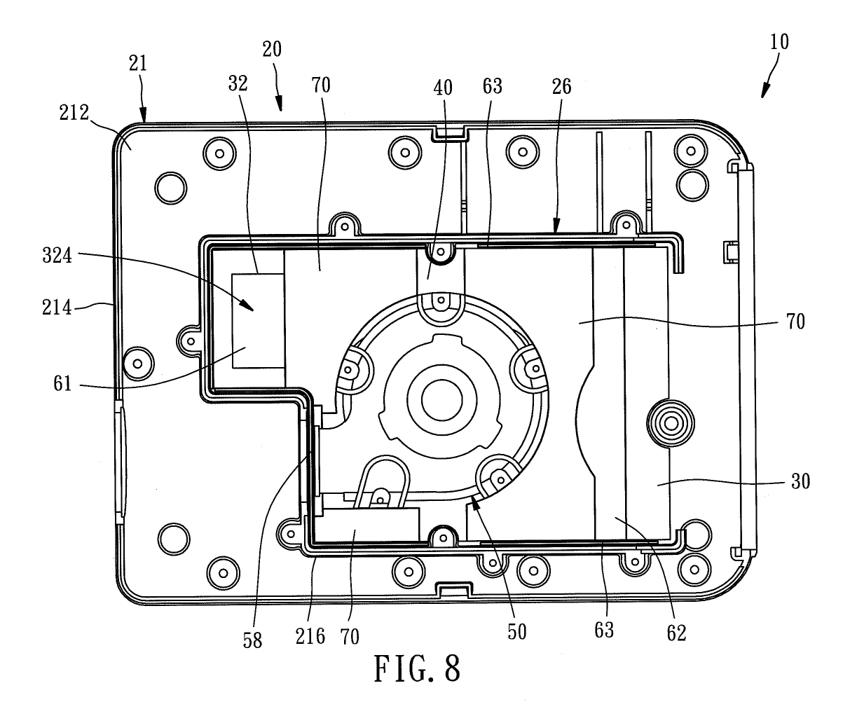

Referring to FIGS. 7-8, the air blower 50 includes a case 52 and a motor 54 mounted inside the case 52. The case 52 is provided with an inlet 56 and an outlet 58. The motor 54 can make the gas be sucked into the case 52 through the inlet 56 and then exhaust, with a certain pressure, through the outlet 58. The air blower 50 is mounted inside the internal space and makes its bottom be engaged with the through hole 42 to be mounted onto the shock-resistant intermediate plate 62. In this way, the inlet 56 can communicate with the exit 326 through the orifice 622. Besides, third sound-absorbing members 70 wrap the air blower 50 and are mounted to the second sound-absorbing member 40.

Referring to FIGS. 4-8 again, while the respirator 10 of the present invention is being operated, the oxygen or air is inputted into the internal space through the first or second intake passage 27 or 28 and then enters the air blower 50 along the channel 32 through the entrance 324 subject to suction of the air blower 50; next, the air blower 50 provides a certain pressure to output the oxygen or air to the external space outside the housing 20 through an outtake passage 29 (FIG. 1) from the outlet 58 and further to the patient's nasal or full-face mask to assist the patient in breathing.

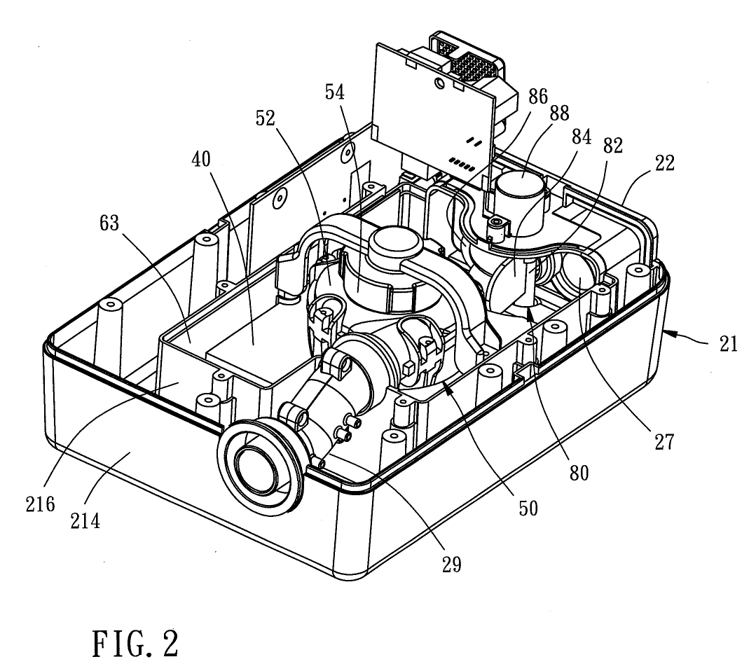

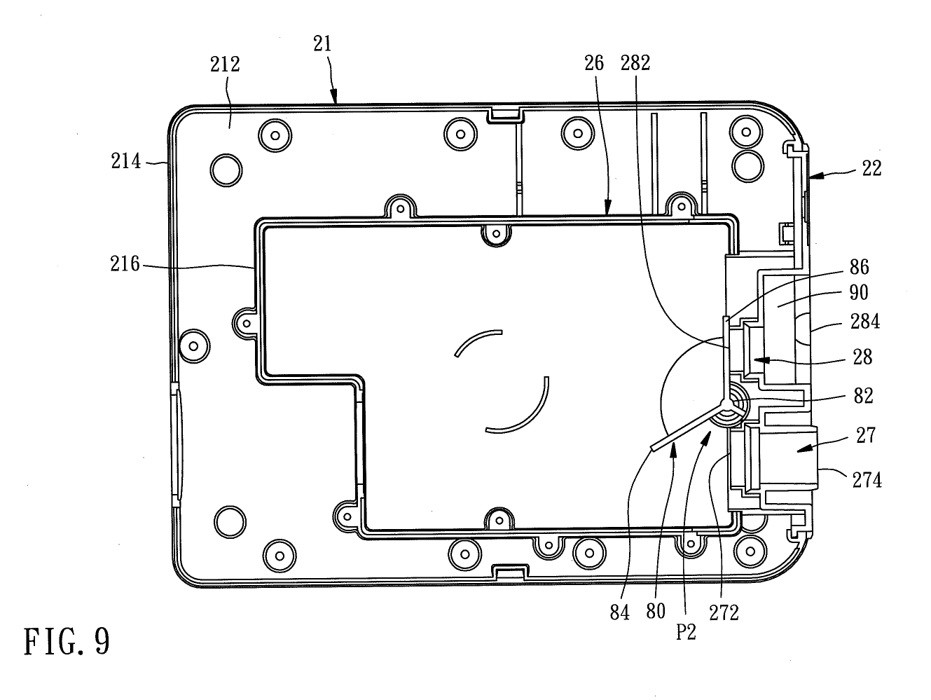

Specifically, the first and second intake passages 27 and 28 can provide the user of the respirator with two kinds of gases as required, i.e. oxygen and air. The housing 20 further includes a rotary valve 80 mounted close to the first and second inner openings 272 and 282. The rotary valve 80 has a rotary shaft 82, a first valve piece 84, and a second valve piece 86, the latter two of which integrally extend toward different directions. The rotary shaft 82 is pivoted between the lower and rear shell pieces 21 and 22 and can be driven for pivoting movement by a motor 88 (FIG. 2) mounted to the rear shell piece 22. When the rotary valve 80 pivots to a first position P1 shown in FIG. 4, the first valve piece 84 fully blocks the first inner opening 272 to prevent the first intake passage 27 from communicating with the internal space; meanwhile, the second intake passage 28 communicates with the internal space, so the respirator 10 can supply the air to the user. When the rotary valve 80 pivots to a second position P2 shown in FIG. 9, the second valve piece 86 fully blocks the second inner opening 282 to prevent the second intake passage 28 from communicating with the internal space; meanwhile, the first intake passage 27 communicates with the internal space, so the respirator 10 can supply oxygen to the user.

In addition, a filter 90 can be further mounted between the second inner and outer openings 282 and 284 of the second intake passage 28. The filter 90 is made of a material through which the gas can pass and which can filter impurities in the gas. For example, the filter 90 can be, but not limited to, a high efficiency particulate air (HEPA) filter. In light of this, when the rotary valve 80 is located at the first position P1, the air outside the housing 20 can be filtered by the filter 90 and then enter the internal space through the second intake passage 28 to allow the respirator 10 to supply fresh air.

When the respirator 10 is being operated, the first sound-absorbing member 30 can absorb the noise generated by the air blower while it extracts the gas, especially the first and second toothed portions 322 and 323 of the channel 32, which can reduce acoustic reflection, so the noise can be decreased. Besides, the second and third sound-absorbing members 40 and 70 can also absorb the noise of the motor 54 in operation and then by the covering the shock-resistant plates 61-63 and the external and internal shell assemblies 25 and 26, the noise inside the internal space of the internal shell assembly 26 conveyed to the external space can be greatly reduced. Thus, less noise is the advantage of the respirator 10. Furthermore, the housing 20 can concentrate the gas, which is to be pressurized and transmitted by the air blower 50, on the internal shell assembly 26, so the efficiency of the gas extraction of the air blower 50 is enhanced to decrease the thermal energy generated by the air blower 50 in such a way that the temperature of the respirator 10 is not subject to rise.

It is to be noted that the primary effects of the present invention are less noise and less temperature rise and can be reached by that the external and internal shell assemblies 25 and 26 cover the noise inside the internal space, the channel 32 formed of the first sound-absorbing member 30 absorbs the noise of the gas extraction of the air blower 50, and the internal shell assembly 26 concentrates the gas, which is to be pressurized and transmitted by the air blower 50, to decrease the thermal energy of the air blower 50. Under the circumstances, the second and third sound-absorbing members 40 and 70 and the shock-resistant plates 61-63 can be excluded from the respirator 10. The present invention can reach another effect of switching gas type by that the first and second intake passages 27 and 28 supply two kinds of gases and the gases can be switched by the rotary valve 80, so the housing 20 of the respirator 10 is not limited to the structure of the preferred embodiment as mentioned above as long as the housing 20 can have any space for saving gas and any two intake passages for communication with the space. Moreover, the second intake passage 28 can directly communicate with a device of supplying fresh air, like air cleaner, so the filter 90 can be excluded from the second intake passage 28.

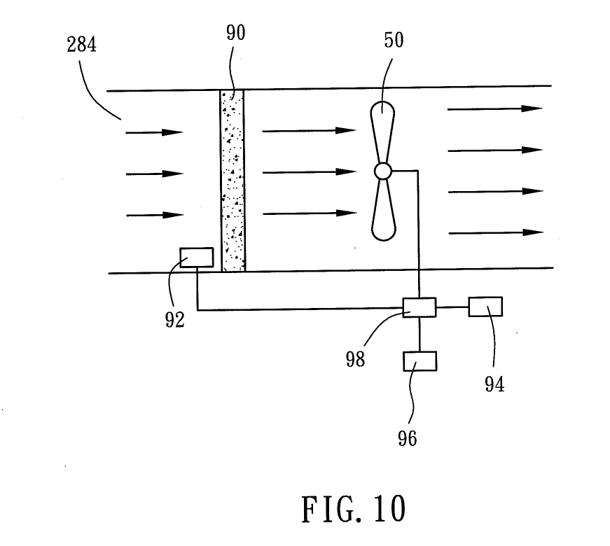

However, if the filter 90 is mounted to the second intake passage 28, the filter 90 will need cleaning or replacement after being used for a period of time. In light of this, the respirator 10 can further include a filter cleanliness/turbidity identification system for enabling the user to know when the filter 90 needs cleaning or replacement. As shown in FIG. 10, the system is composed of a suspended particle sensor 92, a timer 94, a display 96, and a controller 98. The suspended particle sensor 92 is mounted between the filter 90 and the second outer opening 284 for detecting concentration of suspended particles in the external air. The timer 94 can compute the working time of the air blower 50, such as cumulative working fours, working hours for each booting of the air blower 50, or cumulative hours of usage of the filter 90. In this embodiment, the display 96 is mounted to the upper shell piece 24, as shown in FIG. 1, and has three indicators indicative of “GOOD”, “AVERAGE”, and “BAD” for cleanliness/turbidity of the filter 90 separately. Interchangeably, the display 96 can be a screen capable of showing text or a device capable of giving off sound. The controller 98 is electrically connected with the suspended particle sensor 92, the air blower 50, the timer 94, and the display 96 separately for receiving the data detected by the suspended particle sensor 92 and the timer 94 and for controlling the display 96 based on the data.

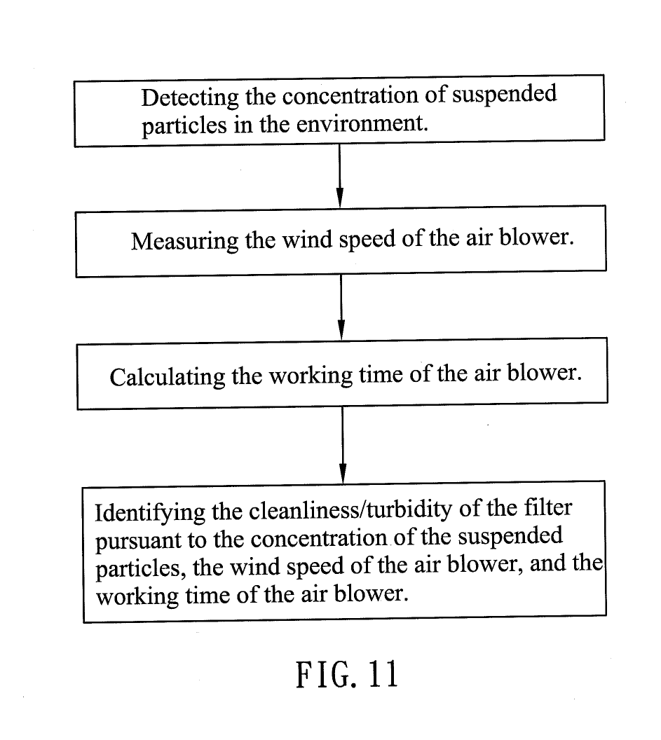

As shown in FIG. 11, a method of identifying the cleanliness/turbidity of the filter can be carried out by the respirator 10 based on the aforesaid filter cleanliness/turbidity identification system, having the following steps.

A) Detect the concentration of suspended particles in the external environment. As the concentration of the suspended particles is higher, the frequency of cleaning or replacing the filter 90 will be higher.

B) Measure the wind speed of the air blower 50. As the wind speed of the air blower 50 is higher, the air passing through the filter 90 within unit time will be more, so the frequency of cleaning or replacing the filter 90 will be higher. Besides, the wind speed of the air blower 50 is related to the wind pressure of the same, so measuring the wind speed of the air blower 50 can be replaced by measuring the wind pressure of the same.

C) Calculate the working time of the air blower 50. As the working hours of the air blower 50 are more to indicate that the filter 90 was used for longer time, it will be more necessary to clean or replace the filter 90. According to different judgment conditions set, the timer can 94 can provide cumulative working hours of the air blower 50, working hours of each booting, cumulative working hours of the filter 90, etc.

D) Identify the cleanliness/turbidity of the filter 90 pursuant to the concentration of the suspended particles in the external environment, the wind speed of the air blower 50, and the working time of the air blower 50. Because the results acquired from the steps A-C are closely related to the cleanliness/turbidity of the filter 90, those results are transmitted to the controller 98 to be treated as judgment parameters and then computed together with a predetermined function to come up with the cleanliness/turbidity of the filter 90. It is to be noted that the purposes of the steps A-C are for acquiring the aforesaid parameters and thus when the present invention is executed, the sequence of the steps A-C is not limited to that of the embodiment of the present invention.

Last but not the least, the controller 98 can display the cleanliness/turbidity of the filter 90 on the display 96 for the user to decide whether to clean or replace the filter 90. Alternatively, the controller 98 can identify the cleanliness/turbidity of the filter 90 and then decide whether it is necessary to clean or replace the filter 90 to generate a signal indicating that the filter 90 is too turbid to need cleaning or replacement and to display the signal on the display 96.

In conclusion, the respirator 10 can not only provide gas types that the user requires but provide filtered fresh air for the user, while the user selects the air, and enable the user to know whether the filter 90 should be replaced to further ensure the cleanliness of the air that the user breathes.

Although the present invention has been described with respect to a specific preferred embodiment thereof, it is in no way limited to the specifics of the illustrated structures but changes and modifications may be made within the scope of the appended claims.

What is claimed is:

1. A respirator comprising:
a housing having an external shell assembly defining an external space formed outside, an internal shell assembly mounted inside the external shell assembly and defining an internal space formed therein, and a first intake passage communicating with the internal and external spaces; and
a first sound-absorbing member mounted inside the internal shell assembly and defining a channel communicating with the internal space.

2. The respirator as defined in claim 1, wherein the channel comprises a first toothed portion.

3. The respirator as defined in claim 2 further comprising a shock-resistant intermediate plate, which is mounted onto the first sound-absorbing member, whereby the channel comprises an entrance and an exit and the first toothed portion is located at the exit.

4. The respirator as defined in claim 3, wherein the channel further comprises a second toothed portion close to the exit of the channel.

5. The respirator as defined in claim 1, wherein the internal shell assembly further comprises an air blower; the first sound-absorbing member communicates with an inlet of the air blower.

6. The respirator as defined in claim 5 further comprising a second sound-absorbing member, which covers the air blower.

7. The respirator as defined in claim 6, wherein the internal shell assembly comprises a bottom, onto which a shock-resistant bottom plate, the first sound-absorbing member, a shock-resistant intermediate plate, and the second sound-absorbing member are mounted; the air blower is mounted to the shock-resistant intermediate plate.

8. The respirator as defined in claim 1, wherein the internal shell assembly comprises a shock-resistant standing plate mounted to one side thereof.

9. The respirator as defined in claim 1, wherein the housing further comprises a second intake passage communicating with the internal and external spaces; the housing further comprises a rotary valve, which is pivotable between a first position and a second position; when the rotary valve is located at the first position, the rotary valve prevents the first intake passage from communicating with the internal space; when the rotary valve is located at the second position, the rotary valve prevents the second intake passage from communicating with the internal space.

10. The respirator as defined in claim 9, wherein the first intake passage comprises a first inner opening facing the internal space; the second intake passage comprises a second inner opening facing the internal space; the rotary valve comprises a rotary shaft, a first valve piece, and a second valve piece, both of which are connected with the rotary shaft; when the rotary valve is located at the first position, the first valve piece fully blocks the first inner opening; when the rotary valve is located at the second position, the second valve piece blocks the second inner opening.

11. The respirator as defined in claim 10 further comprising a motor, which is mounted to the housing and connected with the rotary shaft for driving the rotary valve for pivoting movement.

12. The respirator as defined in claim 9, wherein the second intake passage comprises a second inner opening and a second outer opening, the second inner opening facing the internal space, the second outer opening facing the external space, a filter being mounted between the second inner and outer openings.

13. The respirator as defined in claim 12 further comprising an air blower, a suspended particle sensor, and a controller, wherein the air blower is mounted inside the internal space, the suspended particle sensor is mounted between the filter and the second outer opening, and the controller is electrically connected with the suspended particle sensor.

14. A method of identifying the cleanliness/turbidity of the filter of the respirator defined in claim 13, comprising steps of:
A) detecting the concentration of suspended particles in the environment;
B) measuring the wind speed of the air blower;
C) calculating the working time of the air blower; and
D) identifying the cleanliness/turbidity of the filter pursuant to the concentration of the suspended particles, the wind speed of the air blower, and the working time of the air blower.

15. The method as defined in claim 14 further comprising a step of displaying whether it is necessary or not to clean or replace the filter.
